(19) United States
(12) Patent Application Publication
(10) Pub. No.: US 2023/0263979 A1
(43) Pub. Date: Aug. 24, 2023

(54) ANESTHESIA MACHINE, ANESTHESIA CONTROL METHOD AND ANESTHESIA CONTROL SYSTEM

(71) Applicant: SHENZHEN MINDRAY BIO-MEDICAL ELECTRONICS CO., LTD., Shenzhen (CN)

(72) Inventors: Congying WAN, Shenzhen (CN); Yong HUANGFU, Shenzhen (CN); Chenghua HUANG, Shenzhen (CN); Ruiling PAN, Shenzhen (CN); Xiaoyong ZHOU, Shenzhen (CN)

(73) Assignee: SHENZHEN MINDRAY BIO-MEDICAL ELECTRONICS CO., LTD., Shenzhen (CN)

(21) Appl. No.: 18/113,025

(22) Filed: Feb. 22, 2023

(30) Foreign Application Priority Data
Feb. 23, 2022 (CN) 202210168566.X

(51) Int. Cl. A61M 16/00; A61M 5/172; A61M 16/10

(52) U.S. Cl. CPC A61M 16/024; A61M 5/172; A61M 16/104; A61M 2205/3569; A61M 2205/80; A61M 2205/505; A61M 2205/3584; A61M 2205/3553; A61M 2205/18

(57) ABSTRACT

An anesthesia machine, anesthesia control method and system are provided. The anesthesia machine controls the infusion device, or the control terminal controls the anesthesia machine and infusion device, such that the doctor controls the inhalation and intravenous infusion of anesthetics for patient with only one device, without walking back and forth between multiple devices, which improves the efficiency of anesthesia control.

### CROSS-REFERENCE TO RELATED APPLICATIONS

This application claims priority to Chinese patent Application No. 202210168566.X, filed on Feb. 23, 2022, the content of which is incorporated herein by reference in its entirety.

### TECHNICAL FIELD

The disclosure relates to the technical field of medical device, and more particularly to an anesthesia machine, an anesthesia control method and an anesthesia control system.

### BACKGROUND

In traditional anesthesia process, doctors need to operate an anesthesia machine and an injection pump respectively to complete ventilation maintenance and anesthesia control during surgery. The anesthesia machine is connected with airway of a patient through a respiratory pipeline to deliver a gas with certain pressure and flow rate to the patient to maintain oxygenation of the patient. At the same time, it can also deliver anesthetic gas of a certain concentration. The injection pump is connected with a vein channel of the patient through an infusion pipeline, and delivers a variety of medications to the patient at a certain rate, including sedatives, analgesics, muscle relaxants, vasoactive drugs, etc. In order to maintain the stability of vital signs of the patient during the surgery, the doctor needs to observe monitoring parameters of the vital signs of the patient, and then turn to the anesthesia machine to adjust inhalation of anesthetic and ventilation, or turn to the injection pump to adjust intravenous medication. The doctor switches the workflow between multiple devices, which affects the efficiency and increases the potential risk of misjudgment and mis-operation.

However, positions of the devices in the operating room are not fixed, which increases the complexity of the workflow of the doctor. The anesthesia machine is usually fixed near head of the patient to facilitate the connection of the ventilation pipeline, while the monitor is generally placed next to the anesthesia machine, and the injection pump may be placed at different positions at a body side of the patient according to the different types of surgery. When the injection pump and the anesthesia machine are not in the same place, the doctor needs to move back and forth in the operating room to complete the anesthetic process of monitoring and adjustment. The space in the operating room is generally very crowded, and frequent movement may affect the operation.

Therefore, the efficiency of the existing anesthesia control needs to be improved.

### SUMMARY

In this regard, an anesthesia machine, an anesthesia control method and an anesthesia control system, are provided, aiming at improving the efficiency of anesthesia control.

An embodiment provides an anesthesia control method, including:

establishing communication connection between an anesthesia machine and an infusion device;

controlling, through the anesthesia machine, ventilation of a patient according to anesthesia machine control information which is inputted by a user;

sending, through the anesthesia machine, a corresponding infusion device control instruction to the infusion device according to infusion device control information which is inputted by the user; and

through the infusion device, receiving the infusion device control instruction from the anesthesia machine and controlling medication administration to the patient according to the infusion device control instruction.

In an anesthesia control method provided by an embodiment, the anesthesia machine control information includes: anesthesia machine control information which is inputted by hand and/or anesthesia machine control information which is inputted by voice; the infusion device control information includes infusion device control information which is inputted by hand and/or infusion device control information which is inputted by voice.

In an anesthesia control method provided by an embodiment, the communication connection is wireless communication connection or local area network connection.

In an anesthesia control method provided by an embodiment, the communication connection is wireless communication connection and the anesthesia control method further includes:

through the anesthesia machine, indicating that the wireless communication connection with the infusion device is established, after establishing the wireless communication connection between the anesthesia machine and the infusion device; and/or

through the infusion device, indicating that, the wireless communication connection with the anesthesia machine is established, after establishing the wireless communication connection between the anesthesia machine and the infusion device.

In an anesthesia control method provided by an embodiment, the anesthesia control method further includes:

through the infusion device, indicating that the infusion device control instruction is received, after receiving the infusion device control instruction.

In an anesthesia control method provided by an embodiment, the communication connection is wireless communication connection and the anesthesia control method further includes:

through the anesthesia machine, determining whether information of the patient in the anesthesia machine is consistent with information of the patient in the infusion device, before sending the corresponding infusion device control instruction to the infusion device; if not, disconnecting the wireless communication connection, refusing to send the infusion device control instruction or sending an alarm massage; or

through the infusion device, determining whether information of the patient to whom the infusion device administers medication is consistent with information of the patient whose ventilation is controlled by the anesthesia machine, before controlling the medication administration to the patient according to the infusion device control instruction; if not, disconnecting the wireless communication connection, refusing to receive or respond to the infusion device control instruction or sending an alarm massage.

In an anesthesia control method provided by an embodiment, the anesthesia control method further includes:

through the infusion device, receiving an instruction to close a remote control function and responding to said instruction by stopping receiving the infusion device control instruction or by stopping responding to the infusion device control instruction.

In an anesthesia control method provided by an embodiment, the infusion device includes a first injection pump and a second injection pump; and the anesthesia control method further includes:

through the first injection pump, acquiring a working state of the second injection pump when the first injection pump is in a no-output state or a standby state, and before injection, updating information of the patient in the first injection pump at least based on the acquired working state of the second injection pump.

In an anesthesia control method provided by an embodiment, the infusion device includes a first injection pump; and the anesthesia control method further includes:

through the first injection pump, acquiring a working state of the anesthesia machine when the first injection pump is in a no-output state or a standby state, and before injection, updating information of the patient in the first injection pump at least based on the acquired working state of the anesthesia machine.

In an anesthesia control method provided by an embodiment, the anesthesia control method further includes:

through the anesthesia machine, acquiring a working state of the infusion device when the anesthesia machine is in a no-output state or a standby state, and before ventilation, updating information of the patient in the anesthesia machine at least based on the acquired working state of the infusion device.

In an anesthesia control method provided by an embodiment, the anesthesia control method further includes: displaying, on a same interface of the anesthesia machine, an anesthesia machine setting area for receiving the anesthesia machine control information which is inputted by the user and an infusion device setting area for receiving the infusion device control information which is inputted by the user.

An embodiment provides an anesthesia control method, including:

establishing communication connection between a control terminal and an anesthesia machine, and communication connection between the control terminal and an infusion device;

through the control terminal, sending an anesthesia machine control instruction to the anesthesia machine according to anesthesia machine control information which is inputted by a user, and sending an infusion device control instruction to the infusion device according to infusion device control information which is inputted by the user;

controlling, through the anesthesia machine, ventilation of a patient according to the anesthesia machine control instruction; and

controlling, through the infusion device, medication administration to the patient according to the infusion device control instruction.

In an anesthesia control method provided by an embodiment, the anesthesia machine control information includes: anesthesia machine control information which is inputted by hand and/or anesthesia machine control information which is inputted by voice; the infusion device control information includes infusion device control information which is inputted by hand and/or infusion device control information which is inputted by voice.

In an anesthesia control method provided by an embodiment, the communication connection is wireless communication connection or local area network connection.

In an anesthesia control method provided by an embodiment, the communication connection is wireless communication connection, the control terminal, the anesthesia machine and the infusion device form an anesthesia control system, and the anesthesia control method further includes:

sending connection indication information after wireless communication connection is established between at least one of the control terminal, the anesthesia machine and the infusion device, and other device(s) in the anesthesia control system.

In an anesthesia control method provided by an embodiment, the anesthesia control method further includes:

through the anesthesia machine, indicating that the anesthesia machine control instruction is received after receiving the anesthesia machine control instruction; and/or

through the infusion device, indicating that the infusion device control instruction is received after receiving the infusion device control instruction.

In an anesthesia control method provided by an embodiment, the communication connection is wireless communication connection and the anesthesia control method further includes:

through the control terminal, determining whether information of the patient in the anesthesia machine is consistent with information of the patient in the control terminal, before sending the anesthesia machine control instruction to the anesthesia machine; if not, disconnecting the wireless communication connection, refusing to send the anesthesia machine control instruction or sending an alarm massage; or

through the control terminal, determining whether information of the patient in the infusion device is consistent with information of the patient in the control terminal, before sending the infusion device control instruction to the infusion device; if not, disconnecting the wireless communication connection, refusing to send the infusion device control instruction or sending an alarm massage; or

through the anesthesia machine, determining whether information of the patient in the control terminal is consistent with information of the patient whose ventilation is controlled by the anesthesia machine, before controlling the ventilation of the patient according to the anesthesia machine control instruction; if not, disconnecting the wireless communication connection, refusing to receive or respond to the anesthesia machine control instruction or sending an alarm massage; or

through the infusion device, determining whether information of the patient to whom the infusion device administers medication is consistent with information of the patient in the control terminal, before the controlling the medication administration to the patient according to the infusion device control instruction; if not, disconnecting the wireless communication connection, refusing to receive or respond to the infusion device control instruction or sending an alarm massage.

In an anesthesia control method provided by an embodiment, the anesthesia control method further includes:

through the anesthesia machine, receiving an instruction to close a remote control function and responding to said instruction by stopping receiving the anesthesia machine control instruction or by stopping responding to the anesthesia machine control instruction; and/or

through the infusion device, receiving an instruction to close a remote control function and responding to said instruction by stopping receiving the infusion device control instruction or by stopping responding to the infusion device control instruction.

In an anesthesia control method provided by an embodiment, the infusion device includes a first injection pump and a second injection pump; and the anesthesia control method further includes:

through the first injection pump, acquiring a working state of the second injection pump when the first injection pump is in a no-output state or a standby state, and before injection, updating information of the patient in the first injection pump at least based on the acquired working state of the second injection pump.

In an anesthesia control method provided by an embodiment, the infusion device includes a first injection pump; and the anesthesia control method further includes:

through the first injection pump, acquiring a working state of the anesthesia machine when the first injection pump is in a no-output state or a standby state, and before injection, updating information of the patient in the first injection pump at least based on the acquired working state of the anesthesia machine.

In an anesthesia control method provided by an embodiment, the anesthesia control method further includes:

through the anesthesia machine, acquiring a working state of the infusion device when the anesthesia machine is in a no-output state or a standby state, and before ventilation, updating information of the patient in the anesthesia machine at least based on the acquired working state of the infusion device.

In an anesthesia control method provided by an embodiment, the anesthesia control method further includes: displaying, on a same interface of the control terminal, an anesthesia machine setting area for receiving the anesthesia machine control information which is inputted by the user and an infusion device setting area for receiving the infusion device control information which is inputted by the user.

An embodiment provides an anesthesia control method, including:

establishing communication connection between an anesthesia machine and an infusion device;

setting, through the anesthesia machine, a ventilation parameter according to anesthesia machine control information which is inputted by a user;

sending, through the anesthesia machine, a corresponding infusion device control instruction to the infusion device according to the ventilation parameter; and

through the infusion device, receiving the infusion device control instruction from the anesthesia machine and controlling medication administration to a patient according to the infusion device control instruction.

An embodiment provides an anesthesia control method, including:

establishing communication connection between an anesthesia machine and an infusion device;

controlling, through the infusion device, medication administration to a patient according to infusion device control information which is inputted by a user;

sending, through the infusion device, a corresponding anesthesia machine control instruction to the anesthesia machine according to anesthesia machine control information which is inputted by the user; and

through the anesthesia machine, receiving the anesthesia machine control instruction from the infusion device and controlling ventilation of the patient according to the anesthesia machine control instruction.

An embodiment provides an anesthesia control system, including an anesthesia machine and an infusion device;

wherein the anesthesia machine is configured to communicate with the infusion device, control ventilation of a patient according to anesthesia machine control information which is inputted by a user, send a corresponding infusion device control instruction to the infusion device according to infusion device control information which is inputted by the user; and

the infusion device is configured to receive the infusion device control instruction from the anesthesia machine and control medication administration to the patient according to the infusion device control instruction.

An embodiment provides an anesthesia control system, including a control terminal, an anesthesia machine and an infusion device;

wherein the control terminal is configured to wirelessly communicate with the anesthesia machine and with the infusion device, display an anesthesia machine setting area at a displayer of the control terminal and send an anesthesia machine control instruction to the anesthesia machine according to anesthesia machine control information which is inputted by a user at the anesthesia machine setting area, and display an infusion device setting area at the displayer of the control terminal and send an infusion device control instruction to the infusion device according to infusion device control information which is inputted by the user at the infusion device setting area;

the anesthesia machine is configured to control ventilation of a patient according to the anesthesia machine control instruction; and

the infusion device is configured to control medication administration to the patient according to the infusion device control instruction.

An embodiment provides an anesthesia machine, including:

an anesthetic delivery device, which is configured to mix a gas that is provided by a gas source with an anesthetic and deliver the mixed gas to a respiratory line;

the respiratory line;

a processor, which is configured to control the respiratory line to periodically deliver the mixed gas to a patient to provide anesthesia respiratory support for the patient;

wherein the anesthesia machine further includes:
a communication module, which is in communication connection with an infusion device;
a human-machine interaction device, which is configured to output visual information and receive information which is inputted by a user;
wherein the processor is further configured to receive anesthesia machine control information which is inputted by the user through the human-machine interaction device and control ventilation of the patient according to the anesthesia machine control instruction; and to send a corresponding infusion device control instruction to the infusion device through the communication module, wherein the infusion device control instruction is configured to control the infusion device to administer medication to the patient.

According to the anesthesia machine, anesthesia control method and system of the above embodiments, the anesthesia machine controls the infusion device, or the control terminal controls the anesthesia machine and infusion device, such that the doctor can control the inhalation and intravenous infusion of anesthetic for patient with only one device, without walking back and forth between multiple devices, which improves the efficiency of anesthesia control.

### BRIEF DESCRIPTION OF THE DRAWINGS

FIG. 1 is a block diagram of an anesthesia control system provided in an embodiment of this disclosure.

FIG. 2 is a block diagram of an anesthesia control system provided in another embodiment of this disclosure.

FIG. 3 is a block diagram of an anesthesia machine provided in an embodiment of this disclosure.

FIG. 4 is a flowchart of an anesthesia control method provided in an embodiment of this disclosure.

FIG. 5 is a flowchart of an anesthesia control method provided in another embodiment of this disclosure.

FIG. 6 is a flowchart of an anesthesia control method provided in another further embodiment of this disclosure.

FIG. 7 is a block diagram of an anesthesia control system provided in another further embodiment of this disclosure.

FIG. 8 is a flowchart of an anesthesia control method of the anesthesia control system shown in FIG. 7.

### DETAILED DESCRIPTION

The technical solutions in example embodiments of the disclosure will be described clearly and completely below with reference to the accompanying drawings. Similar elements in different embodiments use similar element reference numbers associated with them. In the following embodiments, many details are described in order to make this disclosure better understood. However, those skilled in the art can easily realize that some of the features can be omitted under different circumstances, or can be replaced by other elements, materials and methods. In some cases, some operations related to this disclosure are not shown or described in the specification, which is to avoid the core part of this disclosure being submerged by too many descriptions. For those skilled in the art, it is not necessary to describe these relevant operations in detail. They can fully understand the relevant operations according to the description in the specification and general technical knowledge in the art.

In addition, the features, operations or features described in the specification can be combined in any appropriate way to form various embodiments. At the same time, the steps or actions in the method description can also be sequentially exchanged or adjusted in a manner that is obvious to those skilled in the art. Therefore, the various sequences in the description and the drawings are only for the purpose of clearly describing an embodiment, and do not mean that they are necessary, unless otherwise stated that one of the sequences must be followed.

The serial number itself, such as “first” and “second”, is only configured to distinguish the described objects, without any sequence or technical meaning. The “connection” and “linkage” mentioned in this application include direct and indirect connection (linkage) unless otherwise specified.

An anesthesia control system provided by this disclosure can centrally control an anesthesia machine and an injection pump to optimize a complex workflow that the anesthesiologist needs to operate on multiple devices. The system includes one main control device and at least one controlled device. The main control device has an interactive interface for receiving information which is inputted by a user. The main control device includes a communication module to send a corresponding setting instruction to the controlled device. The main control device can also give an indication when a control link between the main control device and the controlled device is coma. The main control device can be the anesthesia machine, and the controlled device can be an injection pump. The main control device can also be an additional third-party device, such as a monitor, computer, tablet, etc. At this time, the controlled device can be the anesthesia machine and the injection pump. The following is a detailed description through several embodiments.

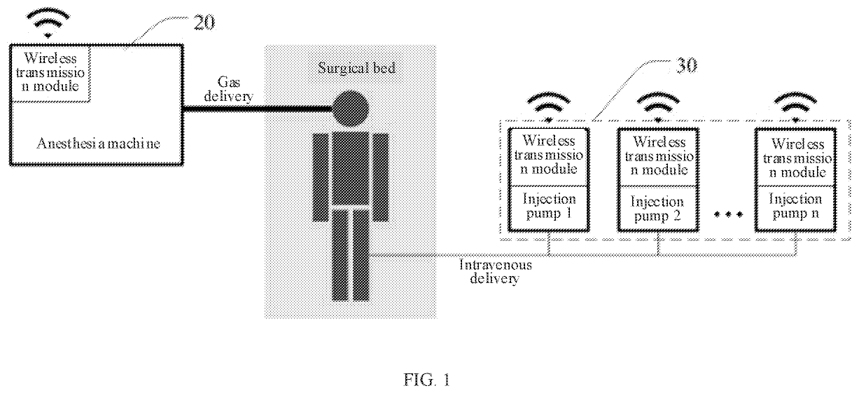

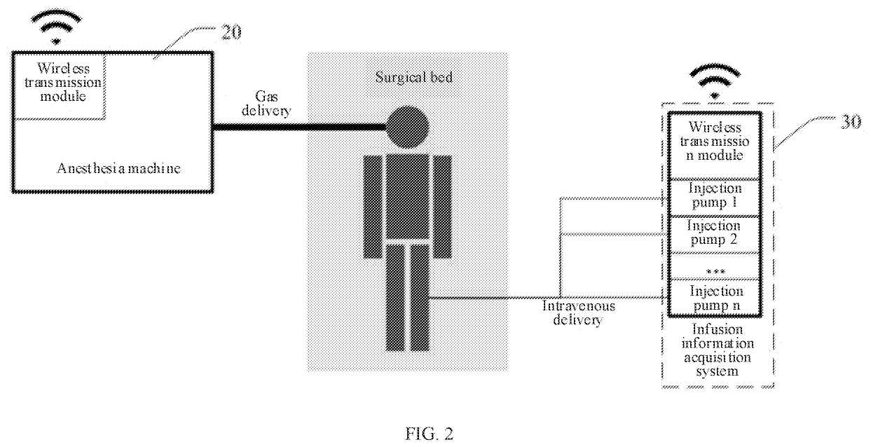

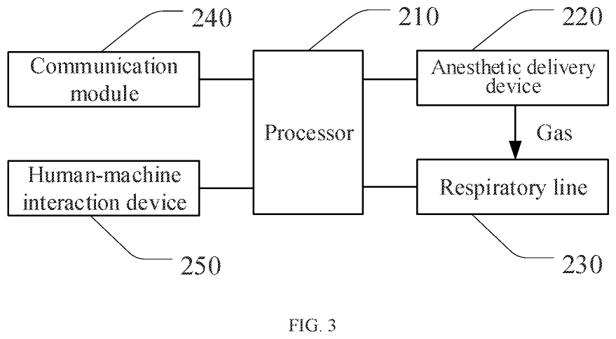

As shown in FIG. 1 and FIG. 2, the anesthesia control system includes an anesthesia machine 20 and an infusion device 30. As shown in FIG. 3, the anesthesia machine 20 includes a processor 210, an anesthetic delivery device 220, a respiratory line 230, a communication module 240 and a human-machine interaction device 250.

The anesthetic delivery device 220 is configured to mix a gas provided by a gas source with an anesthetic and deliver the mixed gas to the respiratory line. The gas source is configured to provide the gas required by the anesthesia machine, such as oxygen, air and nitrous oxide (laughing gas), etc. The gas source can be external, such as various gas cylinders, hospital pipes, etc., or also be internal, such as a turbine. The anesthetic delivery device 220 can use an evaporator.

The respiratory line 230 can be a gas line which connects the anesthetic delivery device 220 and a patient. It can recycle the gas exhaled by the patient to save the anesthetic and reduce environmental pollution. The respiratory line 230 can further include various connection tubes and accessories which can be a nose plug, nose mask, face mask, tracheal tube with a bag at its end, etc. The respiratory line 230 can be provided with a gas purification device for removing at least part of carbon dioxide exhaled by the patient into the respiratory line 230.

The processor 210 is configured to control the respiratory line 230 to periodically deliver the mixed gas to the patient, thus providing anesthesia respiratory support for the patient. For example, the respiratory line 230 is provided with a plurality of valves, and the processor 210 can periodically deliver the mixed gas to the patient through controlling the plurality of valves, so as to provide anesthesia respiratory support for the patient.

The communication module 240 is configured to communicate with other devices in the system, such as the infusion device 30 and the control terminal. The communication module 240 can include a communication interface, which is connected with the infusion device 30 or the control terminal through a cable to realize the communication connection. The communication module 240 can also be a wireless transmission module (that is, a wireless communication module), such as a WIFI module, a WLAM module, a Bluetooth module, and the likes. The wireless transmission module is connected with the infusion device 30 or the control terminal through wireless communication connection.

The human-machine interaction device 250 is for human-machine interaction, for example, it can be configured to output visual information, or receive user input. The human-machine interaction device 250 can use an input apparatus to receive user input, such as keyboard, operation button, mouse, trackball, touch pad, microphone, or touch screen integrated with the display, etc. The human-machine interaction device 250 can output visual information by using displayers, such as various types of displayers.

In an embodiment, as shown in FIGS. 1 and 2, the main control device is the anesthesia machine 20, and the controlled device is the infusion device 30. The anesthesia control system is usually used for one patient, that is, the anesthesia machine 20 and the infusion device 30 work for the same patient.

The processor 210 is further configured to receive anesthesia machine control information which is inputted by the user through the human-machine interaction device 250, for example, to receive the anesthesia machine control information which is inputted by hand or voice of the user through the human-machine interaction device 250, and to control ventilation of the patient according to the anesthesia machine control information. The human-machine interaction device 250 is further configured to receive infusion device control information which is inputted by the user through the human-machine interaction device 250, for example, to receive the infusion device control information which is inputted by hand or voice of the user through the human-machine interaction device 250, and to send a corresponding infusion device control instruction to the infusion device 30 through the communication module 240, wherein the infusion device control instruction is configured to control the infusion device 30 to administer medication to the patient.

The infusion device 30 may include only one injection pump, or multiple (two or more) injection pumps. The infusion device 30 also has a communication module for communicating with the anesthesia machine 20 or the control terminal. The communication module of the infusion device 30 can also be a communication interface or a wireless transmission module. When multiple injection pumps are needed in the surgery, each injection pump can be provided with a separate wireless transmission module (as shown in FIG. 1), or the multiple injection pumps can share a wireless transmission module (as shown in FIG. 2). For example, an infusion information acquisition system is configured to integrate the multiple injection pumps, and the infusion information acquisition system realizes information transmission with the main control device and distributes the received information to the corresponding injection pump. The infusion information acquisition system and the injection pump are connected through a wired or contacted interface, such as a serial port, SPI, I2C, bus, infrared, Bluetooth, etc. The infusion information acquisition system only realizes data transmission and receiving, and the controlled device is still the injection pump.

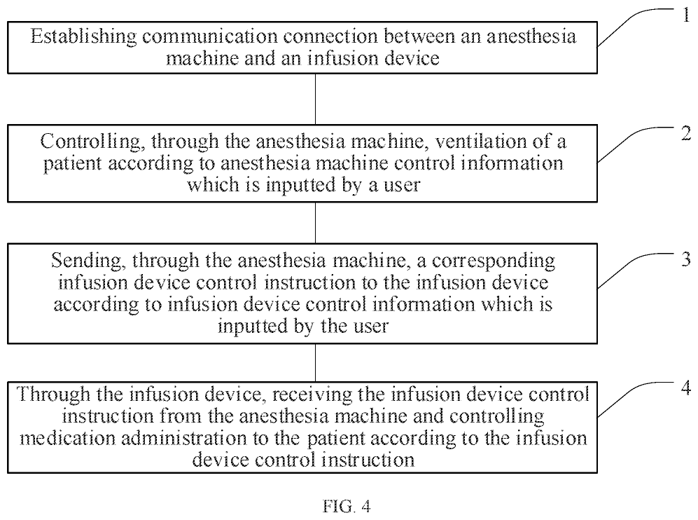

The process of anesthesia control by the anesthesia control system is shown in FIG. 4, including the following steps.

In step 1, communication connection is established between the anesthesia machine 20 and the infusion device 30. The anesthesia machine 20 is connected with the infusion device 30 via wired communication or wireless communication, which can be connected through the LAN. This example takes the wireless communication connection between the two as an example to illustrate. The wireless communication connection requires no cable, the placement of the device is more flexible, and the medical staff does not need to pay attention to the interference of cable during the surgery. The infusion device 30 also has a communication module. This embodiment also takes the wireless transmission module as an example. The wireless transmission module of the anesthesia machine 20 and the wireless transmission module of the infusion device 30 are responsible for establishing data connection between the two. Specifically, the wireless transmission modules of the two devices are paired, and the data connection is established if the pairing is successful.

After the anesthesia machine 20 establishes the wireless communication connection with the infusion device 30, it indicates that a wireless communication connection has been established with the infusion device 30. For example, the anesthesia machine 20 sends first connection indication information to indicate that the wireless communication connection has been established with the infusion device 30. The first connection indication information can be sound indication, light indication, vibration indication, or text and graphic indication on a display interface of the displayer. After the anesthesia machine 20 establishes the wireless communication connection with the infusion device 30, the processor 210 also displays which device is currently connected through the displayer, that is, the processor 210 displays identification of the infusion device 30 which is connected by wireless communication. The identification of the infusion device 30 can be ID, icon or number of the infusion device 30.

Of course, after the infusion device 30 establishes the wireless communication connection with the anesthesia machine 20, it can also indicate that the wireless communication connection has been established with the anesthesia machine 20. For example, the infusion device 30 sends second connection indication information to indicate that the wireless communication connection has been established with the anesthesia machine 20. The second connection indication information can be sound indication, light indication, vibration indication, or text and graphic indication on the display interface of the displayer. After the infusion device 30 establishes the wireless communication connection with the anesthesia machine 20, the displayer of the infusion device 30 also displays which device is currently connected, that is, displays identification of the anesthesia machine 20 which is connected by wireless communication. The identification of the anesthesia machine 20 can be ID, icon or number of the anesthesia machine 20.

In step 2, the anesthesia machine 20 controls ventilation of the patient according to anesthesia machine control information which is inputted by the user. For example, the anesthesia machine 20 controls the ventilation of the patient according to the anesthesia machine control information which is inputted by hand and/or the anesthesia machine control information which is inputted by voice. That is, the user controls the anesthesia machine 20 through the human-machine interaction device 250, and then controls the ventilation of the patient. This embodiment takes the anesthesia machine control information which is inputted by hand of the user as an example for explanation. Specifically, the display interface of the displayer of the anesthesia machine 20 displays an anesthesia machine setting area for receiving the anesthesia machine control information which is inputted by the user. In the anesthesia machine setting area, users can set ventilation control parameters (including ventilation mode, tidal volume or inspiratory pressure, respiratory rate, I:E ratio or inspiratory time, trigger sensitivity, etc.), flowmeter parameters (including oxygen flow, air flow, laughing gas flow, or total flow, oxygen concentration, etc.), electronic evaporator settings (including evaporator selection, output drug concentration, etc.), special function operation (such as inhalation holding, exhalation holding, pulmonary re-expansion), etc. The display interface of the displayer of the anesthesia machine 20 also has an anesthesia information display area, which can display ventilation waveforms (including flow rate, pressure, volume, etc.), ventilation monitoring parameters (including inspiratory/expiratory tidal volume, inspiratory/expiratory minute ventilation volume, respiratory rate, platform pressure, peak pressure, positive end-expiratory pressure, etc.), gas monitoring parameters (inhaled/exhaled anesthetic gas concentration, inhaled/exhaled oxygen concentration, inhaled/exhaled CO₂ concentration, inhaled/exhaled N₂O concentration, etc.). The user can set the anesthesia machine control information in the anesthesia machine setting area based on the displayed information and condition of the patient, so as to control the ventilation of the patient.

In step 3, the anesthesia machine 20 sends a corresponding infusion device control instruction to the infusion device 30 according to infusion device control information which is inputted by the user. For example, the anesthesia machine 20 sends the corresponding infusion device control instruction to the infusion device 30 according to the infusion device control information which is inputted by hand and/or the infusion device control information which is inputted by voice. That is, the processor 210 receives the infusion device control information through the human-machine interaction device 250, then generates the corresponding infusion device control instruction according to the infusion device control information, and sends the corresponding infusion device control instruction to the infusion device 30 through the communication module 240, thus controlling the infusion device 30.

This embodiment takes the infusion device control information which is inputted by hand of the user as an example for explanation. Specifically, the display interface of the displayer of the anesthesia machine 20 displays an infusion device setting area for receiving the infusion device control information which is inputted by the user. In the infusion device setting area, the user can select an active injection pump and set its medication name, medication concentration, injection mode (such as TCI mode, speed mode, body weight mode, etc.), and set injection parameters (such as target control object and target control concentration in TCI mode, injection flow rate in speed mode, waiting volume, etc.). The display interface of the displayer of the anesthesia machine 20 also has an injection information display area. The injection information display area can display an amount of injection, amount to be injected, flow rate, plasma concentration, effect chamber concentration, etc. The user can control the infusion device by setting the infusion device control information in the infusion device setting area based on the displayed information and condition of the patient.

Among them, the anesthesia machine setting area and the infusion device setting area may not appear on the same display interface (interactive interface), that is, the two areas can be displayed on different display interfaces of the anesthesia machine, or can be displayed on the same interface of the anesthesia machine, that is, the two areas can be displayed on the same display interface at the same time. The latter is explained as an example. Users can adjust the parameters of anesthesia machine 20 and infusion device 30 on the same interactive interface, without operating separately on the two devices or walking between the two devices, which improves the work efficiency and safety. Similarly, the anesthesia information display area and the injection information display area may not appear on the same display interface, and may also be displayed on the same interface of the anesthesia machine. The latter is explained as an example in this embodiment. In this embodiment, in order to understand completely and control the situation of the patient, the anesthesia machine setting area, the infusion device setting area, the anesthesia information display area and the injection information display area are displayed on the same display interface. Of course, in some embodiments, various types of physical keys can be configured to replace virtual keys in the anesthesia machine setting area and the infusion device setting area for the user to input control information.

Before the processor 210 sends the corresponding infusion device control instruction to the infusion device 30 through the communication module 240, it can also determine whether information of the patient in the anesthesia machine 20 is consistent with information of the patient in the infusion device 30. If yes, the processor 210 sends the corresponding infusion device control instruction to the infusion device 30 through the communication module 240. Or else, the processor 210 indicates that there is a problem. For the sake of safety, the processor 210 disconnects the wireless communication connection, that is, disconnects the wireless communication connection with the infusion device 30 through the communication module 240, and also refuses to send the infusion device control instruction, and/or sends an alarm massage, such as outputting first alarm information, which can be sound indication, light indication, vibration indication, or text and graphic indication on a display interface of the displayer, to warn the user that the information of the patient is currently abnormal.

In step 4, the infusion device 30 receives the infusion device control instruction from the anesthesia machine 20, and controls medication administration to the patient according to the infusion device control instruction. It can be seen that the medical staff can control the anesthesia machine 20 and the infusion device 30 only by operating the anesthesia machine 20, which is very convenient and efficient.

In the embodiment shown in FIG. 1, the anesthesia machine 20 is the main control device, the injection pump is the controlled device, and the anesthesia machine 20 is information-interconnected with one or more injection pumps through their respective wireless transmission module. The interactive interface is deployed at the anesthesia machine 20. This interface provides not only control operation of the anesthesia machine itself, but also control operation of the injection pump. The user sets, on the interactive interface, setting parameters for delivering intravenous medication to the patient. The interactive interface sends the instructions to the injection pump through the wireless transmission module. The injection pump delivers intravenous medication to patient according to the received instruction.

In the embodiment shown in FIG. 2, the infusion device 30 includes multiple injection pumps and an infusion information acquisition system. The multiple injection pumps are integrated with the infusion information acquisition system and connected with the anesthesia machine 20 through one wireless transmission module. The infusion information acquisition system is connected with the injection pump through wired or contact connection, such as serial port, I2C interface, bus, etc., and is provided with a wireless transmission module to connect with the anesthesia machine. The infusion information acquisition system sends the acquired information of each injection pump to the anesthesia machine, and then forwards the control instruction sent by the anesthesia machine to the corresponding injection pump.

The infusion device 30 indicates that the infusion device control instruction has been received after receiving the infusion device control instruction. For example, the infusion device 30 sends first receiving indication information to indicate that the infusion device control instruction has been received. The first receiving indication information can be one or more of the following: sound indication (such as indication tone), light indication (such as flashing LED light, color change of all or part of the screen of the infusion device 30), vibration indication, identification indication of screen display state of the infusion device 30 (such as text, graphics), etc. The infusion device 30 feeds back the first receiving indication information to the user, which is convenient for the user to confirm that the infusion device 30 has received the control instruction, thus improving the safety of operation.

In the system provided by this disclosure, after the injection pump and the anesthesia machine have successfully established a wireless connection, the anesthesia machine can pop up an indication message of successful connection, and the injection pump can also send an indication message of successful connection. When the user sets control parameters of the injection pump on the interactive interface, the injection pump also sends a corresponding indication message to remind the user that it has received the control parameters after receiving these control parameters. Accordingly, the user can understand completely each step according to the indication messages, which improves the security.

Before the infusion device 30 can determine whether information of the patient to whom the infusion device 30 administers medication is consistent with information of the patient whose ventilation is controlled by the anesthesia machine 20, before the controlling medication administration to the patient according to the infusion device control instruction. If yes, the infusion device 30 can control the medication administration to the patient according to the infusion device control instruction. Or else, the infusion device 30 disconnects the wireless communication connection, that is, disconnects the wireless communication connection with the anesthesia machine 20, and also refuses to receive or respond to the infusion device control instruction, and/or sends an alarm massage, such as outputting second alarm information, which can be sound indication, light indication, vibration indication, or text and graphic indication displayed on the screen, to warn the user that the information of the patient is currently abnormal.

The infusion device 30 also provides an option for whether to accept remote control for the user to set. For example, when the user decides that the infusion device 30 can accept remote control, the user can operate the infusion device 30 to send an instruction to start a remote control function (such as selecting the option of accepting remote control). At this time, the infusion device 30 can receive a remote control instruction and execute it, such as allowing to receive the infusion device control instruction or to respond to the infusion device control instruction. When the user does not want this infusion device 30 to accept the remote control, the user can operate the infusion device 30 to send an instruction to close a remote control function (such as cancelling selection of the option of accepting remote control). The infusion device 30 receives the instruction to close the remote control function, and responds to said instruction by stopping receiving the infusion device control instruction or by stopping responding to the infusion device control instruction. That is, the infusion device 30 is no longer controlled by the anesthesia machine 20, which can avoid subsequent mis-operation.

The infusion device 30 can also determine whether the patient is changed, and process the workflow which is restarted after its standby according to the determination result of whether the patient is changed.

For example, the infusion device 30 includes a first injection pump. Anesthesia machine 20 has three working states: output state (outputting anesthetic to the patient), no-output state (not outputting anesthetic to the patient) and standby state (such as, opening a gripper of injection pump). When the first injection pump is in the no-output state or standby state, the working state of the anesthesia machine 20 is acquired. The first injection pump updates the information of the patient of the first injection pump at least based on the acquired working state of the anesthesia machine 20 before starting injection. Specifically, if the acquired working state of the anesthesia machine 20 is the output state, it means that the first injection pump is in the no-output state or the standby state temporarily, and then it continues to give medication to the original patient when it enters the output state. That is, it is not necessary to input information of patient again after the first injection pump restarts to output. If the acquired working state of anesthesia machine 20 is no-output state or standby state, it can be considered that the infusion device 30 and anesthesia machine 20 have completed the work for the current patient. After the first injection pump restarts to output (the next time it enters the output state), it is necessary to update information of the patient in the first injection pump, and after the anesthesia machine 20 restarts to output, it is also necessary to update the information of the patient.

As another example, the infusion device 30 includes a first injection pump and a second injection pump. The injection pump has three working states: output state (injecting medication to the patient), no-output state (not injecting medication to the patient) and standby state (such as, opening a gripper of injection pump). When the first injection pump is in no-output state or standby state, the working state of the second injection pump is acquired. Before the first injection pump starts to inject, information of the patient in the first injection pump is updated at least based on the acquired working state of the second injection pump. In this embodiment, the first injection pump acquires the working state of other controlled devices (such as the second injection pump and anesthesia machine). Before the first injection pump starts to inject, information of the patient in the first injection pump is updated based on the acquired working state of other controlled devices. Specifically, if the acquired working state of the second injection pump is in the output state, it means that the first injection pump is in the no-output state or the standby state temporarily, and then it continues to give medication to the original patient when it enters the output state. That is, it is not necessary to input information of patient again after the first injection pump restarts to output. If the acquired working state of second injection pump is no-output state or standby state, the working state of the other injection pumps (if any) of the infusion device 30 can also be acquired. If the other injection pumps are also in the no-output state or standby state, it can be considered that the infusion device 30 has completed the work for the current patient. After the first injection pump restarts to output (the next time it enters the output state), it is necessary to update information of the patient in the first injection pump. If multiple injection pumps are used for intravenous administration during the surgery, when the gripper of any injection pump is opened, there are still other injection pumps continuing to inject. At this time, it can be determined that the injection pump with the opened gripper only needs to replace medication, and the replaced medication can still be administered according to the information of the previous patient. In this way, there is no need for users to input or confirm the information of the patient before each injection. If all controlled devices of different types enter the standby state or enter the no-output state during the surgery, such as the ventilation model of the anesthesia machine cannot be monitored, and the injection pump enters the standby state, then it can be determined that the anesthesia of the last patient has ended, and the information of the patient needs to be re-inputted when the next ventilation or injection is restarted.

When the anesthesia machine 20 is in the no-output state or standby state, the working state of the infusion device 30 can also be acquired. The anesthesia machine 20 updates the information of the patient of the anesthesia machine at least based on the acquired working state of the infusion device 30 before starting ventilation. Specifically, if the acquired working state of the infusion device 30 is in the output state, it means that the anesthesia of the patient is not over, and the anesthesia machine 20 continues to give anesthetic to the original patient when it enters the output state, that is, the anesthesia machine 20 does not need to re-input the information of the patient after restarting to output. If the acquired working state of infusion device 30 is in the no-output state or standby state, it can be considered that the infusion device 30 and anesthesia machine 20 have completed the work for the current patient, and the anesthesia machine 20 needs to update the information of the patient after restarting to output.

To sum up, this disclosure integrates the control of anesthesia machine and injection pump, and sends user instruction through remote transmission, which can solve the problem of complex workflow of doctor and the need to switch and operate multiple devices. For remote transmission, although the traditional wired connection can solve the need for centralized control, when the anesthesia machine and injection pump are placed at different sides of the patient, the connection cable may invade the work area of the doctor and cause potential risks. Therefore, wireless connection is the most ideal connection mode, which can realize the centralized control of anesthesia machine and injection pump without introducing additional cable management risks. However, the wireless connection mode may introduce the risk of misoperation. There are usually multiple anesthesia machines and injection pumps in multiple adjacent operating rooms. Inadvertent operation may connect the devices in different operating rooms, which results in that setting instructions of the doctor are sent to unexpected device, thus causing improper medication administration. This disclosure avoids the risk of connecting devices in different operating rooms through wireless pairing, indicating after successful pairing, and indicating after that the controlled device receives the instruction, which improves the efficiency of anesthesia control while ensuring safety.

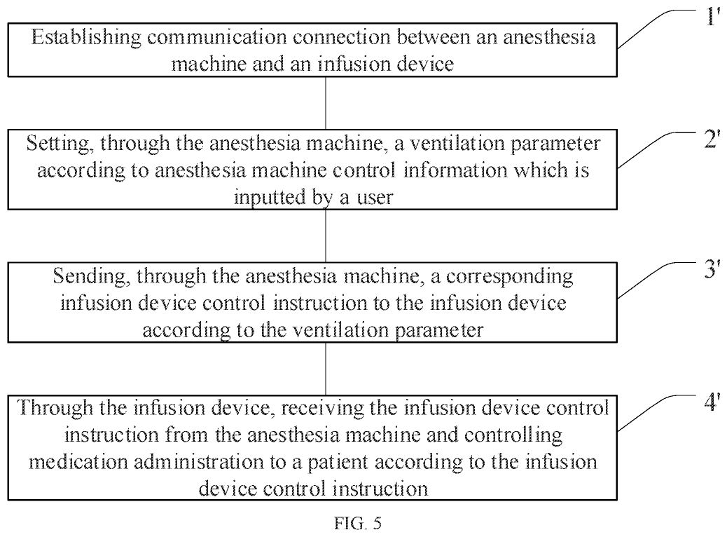

The above steps 2 and 3 are for the user to set various parameters of the anesthesia machine and infusion device on the display interface of the anesthesia machine. In some embodiments, the anesthesia machine can generate corresponding infusion device control instruction according to the anesthesia machine control information. As shown in FIG. 5, the corresponding anesthesia control method includes the following steps.

In step 1′, communication connection is established between the anesthesia machine 20 and the infusion device 30. The specific process is the same as step 1 of the embodiment shown in FIG. 4, and is not repeatedly described here.

In step 2′, the anesthesia machine 20 sets a ventilation parameter according to anesthesia machine control information which is inputted by a user. For example, the ventilation parameter is set according to anesthesia machine control information which is inputted by hand and/or anesthesia machine control information which is inputted by voice. The ventilation parameter includes one or more of the above ventilation control parameters, flow meter parameters, electronic evaporator settings, and special function operation. Then the anesthesia machine 20 controls the ventilation of the patient according to the ventilation parameter. The specific process is shown in step 2 of the embodiment shown in FIG. 4, which is not repeatedly described here.

In step 3′, the anesthesia machine 20 sends the corresponding infusion device control instruction to the infusion device 30 according to the ventilation parameter. For example, the anesthesia machine 20 is preset with a corresponding relationship between different ventilation parameters and infusion device control instructions. The anesthesia machine 20 acquires its corresponding infusion device control instruction according to the current ventilation parameter, and then sends the corresponding infusion device control instruction to the infusion device 30. It can be seen that the anesthesia machine 20 can intelligently adjust the parameters of the infusion device 30 according to the ventilation parameter, thus realizing a certain degree of automatic control of the infusion device 30.

In step 4′, the infusion device 30 receives the infusion device control instruction from the anesthesia machine, and controls medication administration to the patient according to the infusion device control instruction. The specific process is the same as step 4 of the embodiment shown in FIG. 4, and is not repeatedly described here.

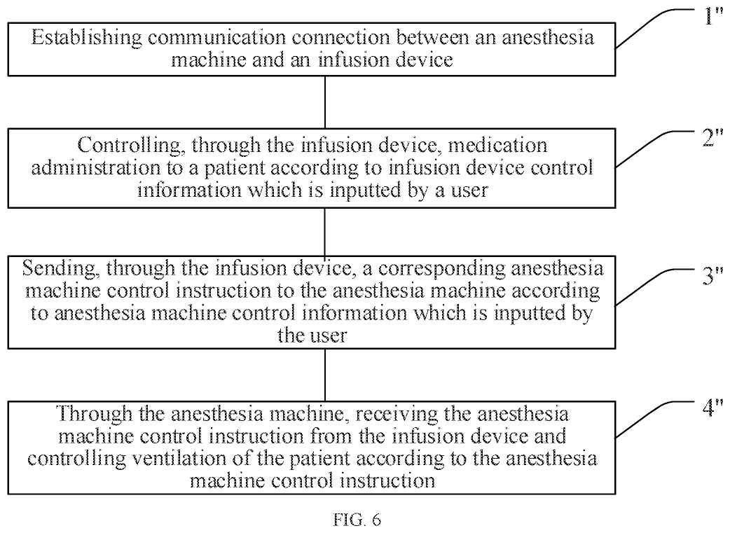

The above embodiments all refer to that the anesthesia machine 20 controls the infusion device 30. However, in some other embodiments, the anesthesia machine 20 can also be controlled by the infusion device 30, that is, infusion device 30 is the main control device, and the anesthesia machine 20 is the controlled device. As shown in FIG. 6, the corresponding anesthesia control method includes the following steps.

In step 1″, communication connection is established between the anesthesia machine 20 and the infusion device 30. The specific process is the same as step 1 of the embodiment shown in FIG. 4, and is not repeatedly described here.

In step 2″, the infusion device 30 controls medication administration to a patient according to infusion device control information which is inputted by a user, such as according to the infusion device control information which is inputted by hand and/or the infusion device control information which is inputted by voice. For example, the infusion device 30 generates a corresponding infusion device control instruction according to the infusion device control information which is inputted by hand and/or by voice, by the user, and then controls the medication administration to the patient according to the infusion device control instruction.

In step 3″, the infusion device 30 sends an anesthesia machine control instruction to the anesthesia machine according to anesthesia machine control information which is inputted by a user, such as according to the anesthesia machine control information which is inputted by hand and/or the anesthesia machine control information which is inputted by voice. For example, the infusion device 30 generates a corresponding anesthesia machine control instruction according to the anesthesia machine control information which is inputted by hand and/or by voice by the user, and then sends the corresponding anesthesia machine control instruction to the anesthesia machine 20.

In step 4″, the anesthesia machine receives the anesthesia machine control instruction from the infusion device, and controls ventilation of the patient according to the anesthesia machine control instruction.

The method shown in FIG. 6 can be acquired by replacing the anesthesia machine in each step of the embodiment shown in FIG. 4 with the infusion device, and replacing the infusion device in each step of the embodiment shown in FIG. 4 with the anesthesia machine. Therefore, the specific process of the embodiment shown in FIG. 6 can refer to the embodiment shown in FIG. 4, with only interchanging the main control device and the controlled device.

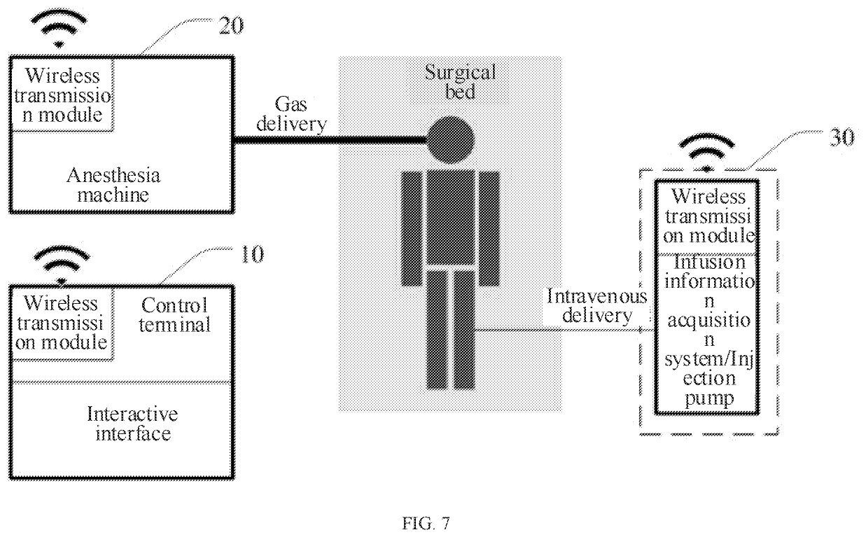

In another embodiment, as shown in FIG. 7, the anesthesia control system includes a control terminal 10, an anesthesia machine 20 and an infusion device 30. The main control device is the control terminal 10, and the controlled devices are the anesthesia machine 20 and the infusion device 30.

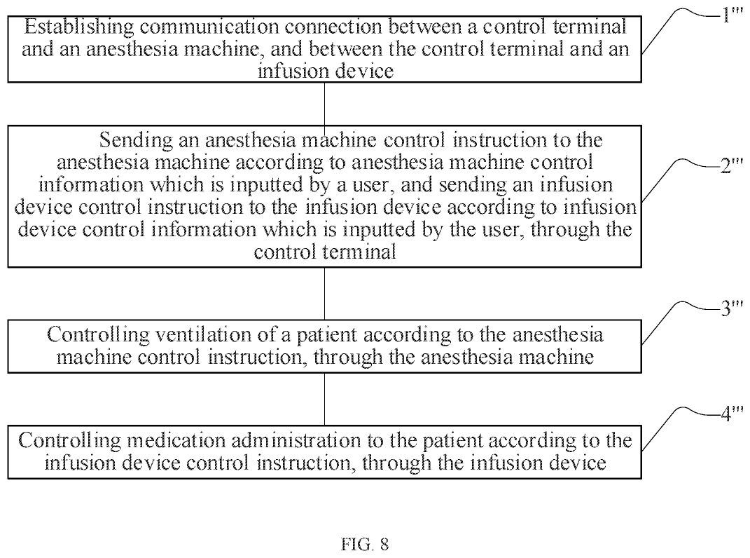

The control terminal 10 can be a monitor, computer, tablet computer, etc. The anesthesia control process of the anesthesia control system shown in FIG. 7 is shown in FIG. 8, including the following steps.

In step 1′″, communication connection is established between the control terminal 10 and the anesthesia machine 20 and between the control terminal 10 and the infusion device 30. Similarly, the control terminal 10 is connected with the anesthesia machine 20 via wired communication or wireless communication, which can be connected through the LAN, and the control terminal 10 is connected with the infusion device 30 via wired communication or wireless communication, which can be connected through the LAN. The control terminal 10 also has a communication module. This embodiment also takes the wireless transmission module as an example. The wireless transmission module of the control terminal 10 is responsible for establishing data connection with the controlled device. Specifically, the wireless transmission module of the control terminal 10 is paired with the wireless transmission module of the controlled device, and the data connection is established if the pairing is successful.

At least one of the control terminal 10, the anesthesia machine 20 and the infusion device 30 sends connection indication information after establishing a wireless communication connection with other devices of the anesthesia control system.

In this embodiment, the control terminal 10 indicates that the wireless communication connection has been established with the anesthesia machine 20 after establishing the wireless communication connection with the anesthesia machine 20. For example, the control terminal 10 sends third connection indication information to indicate that a wireless communication connection has been established with the anesthesia machine 20. After establishing the wireless communication connection with the anesthesia machine 20, the control terminal 10 also displays which device is currently connected through its own displayer, that is, the control terminal 10 displays an identification of the anesthesia machine 20 which is connected by wireless communication. The identification of the anesthesia machine 20 can be ID, icon or number of the anesthesia machine 20.

Similarly, the control terminal 10 indicates that the wireless communication connection has been established with the infusion device 30 after establishing the wireless communication connection with the infusion device 30. For example, the control terminal 10 sends fourth connection indication information to indicate that the wireless communication connection has been established with the infusion device 30. After establishing the wireless communication connection with the infusion device 30, the control terminal 10 also displays which device is currently connected through its own displayer, that is, the control terminal 10 displays identification of the infusion device 30 which is connected by wireless communication.

Of course, the anesthesia machine 20 can also indicates that the wireless communication connection has been established with the control terminal 10 after establishing the wireless communication connection with the control terminal 10. For example, the anesthesia machine 20 sends fifth connection indication information, to indicate that the wireless communication connection has been established with the control terminal 10. After establishing the wireless communication connection with the control terminal 10, the anesthesia machine 20 also displays which device is currently connected through its displayer, that is, the anesthesia machine 20 displays identification of the control terminal 10 which is connected by wireless communication.

Of course, the infusion device 30 can also indicates that the wireless communication connection has been established with the control terminal 10 after establishing the wireless communication connection with the control terminal 10. For example, the infusion device 30 sends sixth connection indication information, to indicate that the wireless communication connection has been established with the control terminal 10. After establishing the wireless communication connection with the control terminal 10, the infusion device 30 also displays which device is currently connected through its displayer, that is, the infusion device 30 displays identification of the control terminal 10 which is connected by wireless communication.

The third connection indication information, the fourth connection indication information, the fifth connection indication information and the sixth connection indication information can be sound indication, light indication, vibration indication, or text and graphic indication on an display interface of the displayer.

In step 2′″, the control terminal 10 sends an anesthesia machine control instruction to the anesthesia machine 20 according to anesthesia machine control information which is inputted by the user, and an infusion device control instruction to the infusion device 30 according to infusion device control information which is inputted by the user. For example, the control terminal 10 sends the anesthesia machine control instruction to the anesthesia machine 20 according to the anesthesia machine control information which is inputted by hand and/or the anesthesia machine control information which is inputted by voice, and sends the infusion device control instruction to the infusion device 30 according to the infusion device control information which is inputted by hand and/or the infusion device control information which is inputted by voice. This embodiment takes the control information which is inputted by hand of the user as an example for explanation. Specifically, the display interface of the displayer of the control terminal 10 displays an anesthesia machine setting area for receiving the anesthesia machine control information which is inputted by the user. The anesthesia machine setting area is the same as the above example, so it is not repeatedly described here. The control terminal 10 generates the corresponding anesthesia machine control instruction according to the anesthesia machine control information, and sends the corresponding anesthesia machine control instruction to the anesthesia machine 20 to control the anesthesia machine 20. The control terminal 10 also acquires information of the anesthesia machine 20. The display interface of the displayer of the control terminal 10 also has an anesthesia information display area. The anesthesia information display area is the same as the above example, so it is not repeatedly described here.

The display interface of the displayer of the control terminal 10 also displays an infusion device setting area for receiving the infusion device control information which is inputted by the user. The infusion device setting area is the same as the above example, so it is not repeatedly described here. The control terminal 10 also acquires information of the infusion device 30. The display interface of the displayer of the control terminal 10 also has an injection information display area. The injection information display area is the same as the above example, so it is not repeatedly described here.

Among them, the anesthesia machine setting area and the infusion device setting area can not appear on the same display interface (interactive interface), that is, the two areas can be displayed on different display interfaces of the control terminal 10, or can appear on the same interface of the control terminal 10, that is, the two areas can be displayed on the same display interface at the same time. Users can adjust the parameters of anesthesia machine 20 and infusion device 30 on the same interactive interface, without operating separately on the two devices or walking between the two devices, which improves the work efficiency and safety. Similarly, the anesthesia information display area and the injection information display area can not appear in the same display interface, and can also be displayed in the same interface of the anesthesia machine. In this embodiment, in order to understand completely and control the situation of the patient, the anesthesia machine setting area, the infusion device setting area, the anesthesia information display area and the injection information display area are displayed on the same display interface.

Before the control terminal 10 sends the corresponding anesthesia machine control instruction to the anesthesia machine, it can also determine whether information of the patient in the control terminal 10 is consistent with information of the patient in the anesthesia machine. If yes, the control terminal 10 sends the corresponding anesthesia machine control instruction to the anesthesia machine. Or else, the control terminal 10 disconnects the wireless communication connection, that is, disconnects the wireless communication connection with the anesthesia machine, and also refuses to send the anesthesia machine control instruction, and/or sends an alarm massage, such as outputting third alarm information.

Before the control terminal 10 sends the corresponding infusion device control instruction to the infusion device 30, it can also determine whether information of the patient in the control terminal 10 is consistent with information of the patient in the infusion device 30. If yes, the control terminal 10 sends the corresponding infusion device control instruction to the infusion device 30. Or else, the control terminal 10 disconnects the wireless communication connection, that is, disconnects the wireless communication connection with the infusion device 30, and also refuses to send the infusion device control instruction, and/or sends an alarm massage, such as outputting the fourth alarm information.

The third alarm information and the fourth alarm information can be sound indication, light indication, vibration indication, or text and graphic indication on the display interface of the displayer to warn the user that the information of the patient is currently abnormal.

In step 3′″, the anesthesia machine 20 controls ventilation of the patient according to the anesthesia machine control instruction. The anesthesia machine 20 indicates that the anesthesia machine control instruction has been received after receiving the anesthesia machine control instruction. For example, the anesthesia machine sends third receiving indication information to indicate that the anesthesia machine control instruction has been received.

Before the anesthesia machine 20 controls ventilation of the patient according to the anesthesia machine control instruction, it determines whether information of the patient in the infusion device is consistent with information of the patient in the control terminal. If yes, the anesthesia machine 20 controls the ventilation of the patient according to the anesthesia machine control instruction. Or else, the anesthesia machine disconnects the wireless communication connection, that is, disconnects the wireless communication connection with the control terminal 10, and also refuses to receive or respond to the anesthesia machine control instruction or sends an alarm massage, such as outputting fifth alarm information.

In step 4′″, the infusion device 30 controls medication administration to the patient according to the infusion device control instruction. The infusion device 30 indicates that the infusion device control instruction has been received after receiving the infusion device control instruction. For example, the infusion device 30 sends fourth receiving indication information to indicate that the infusion device control instruction has been received.

The third receiving indication information and the fourth receiving indication information can also be one or more of the following: sound indication (such as indication tone), light indication (such as flashing LED light, color change of all or part of the screen), vibration indication, identification indication of screen display state (such as text, graphics), etc. The controlled device feeds back receiving indication information to the user, which is convenient for the user to confirm that the controlled device has received the control instruction, thus improving the safety of operation.

Before the infusion device 30 controls the medication administration to the patient according to the infusion device control instruction, it can also determine whether information of the patient to whom the infusion device 30 administers medication is consistent with information of the patient in the control terminal 10. If yes, the infusion device 30 controls the medication administration to the patient according to the infusion device control instruction. Or else, the infusion device 30 disconnects the wireless communication connection, that is, disconnects the wireless communication connection with the control terminal 10, and also refuses to receive or respond to the infusion device control instruction or sends an alarm massage, such as outputting sixth alarm information.

The controlled device also provides an option for whether to accept remote control for the user to set. For example, when the user decides that the anesthesia machine 20 can accept remote control, the user can operate the anesthesia machine 20 to send an instruction to start a remote control function (such as selecting the option of accepting remote control). At this time, the anesthesia machine 20 can receive a remote control instruction and execute it, such as allowing to receive the anesthesia machine control instruction or to respond to the anesthesia machine control instruction. When the user does not want this anesthesia machine 20 to be controlled remotely, the user can operate the anesthesia machine 20 to send an instruction to close a remote control function (such as cancelling selection of the option of accepting remote control). The anesthesia machine 20 receives the instruction to close the remote control function, and responds to said instruction by stopping receiving the anesthesia machine control instruction or by stopping responding to the anesthesia machine control instruction. That is, the anesthesia machine 20 is no longer controlled by the control terminal 10, which can avoid subsequent mis-operation.

Similarly, the infusion device 30 receives an instruction to close a remote control function and responds to said instruction by stopping receiving the infusion device control instruction or by stopping responding to the infusion device control instruction. The specific process can refer to the above embodiments, and is not repeatedly described here.

Similarly, in this embodiment, the infusion device 30 can also determine whether the patient is changed, and process the workflow which is restarted after its standby according to the determination result of whether the patient is changed. For example, the infusion device 30 includes a first injection pump. When the first injection pump is in the no-output state or standby state, the working state of the anesthesia machine 20 is acquired. The first injection pump updates the information of the patient of the first injection pump at least based on the acquired working state of the anesthesia machine 20 before starting injection. The specific process is the same as the above embodiments, and is not repeatedly described here.

As another example, the infusion device 30 includes a first injection pump and a second injection pump. When the first injection pump is in the no-output state or standby state, the working state of the second injection pump is acquired. Before the first injection pump starts injection, information of the patient in the first injection pump is updated at least based on the acquired working state of the second injection pump. The specific process is the same as the above embodiments, and is not repeatedly described here.

When the anesthesia machine 20 is in the no-output state or standby state, the working state of the infusion device 30 (that is, the working state of the injection pump) can also be acquired. The anesthesia machine 20 updates the information of the patient of the anesthesia machine at least based on the acquired working state of the infusion device 30 before starting ventilation. The specific process is the same as the above embodiments, and is not repeatedly described here.

In this embodiment, the control terminal 10 is a tablet computer, that is, the above interactive interface is deployed on a mobile tablet computer. The doctor can place the tablet computer at any suitable position in the operating room according to the surgical needs and the space constraints in the operating room, and send a control instruction to the anesthesia machine and injection pump on the tablet computer. The controlled devices, such as the anesthesia machine and injection pump, are interconnected with the main control device, such as the tablet computer, through the wireless transmission module to receive the control instruction, thus realizing the gas delivery and intravenous medication delivery to the patient, and improving the efficiency of the anesthesia operation.

Various exemplary embodiments are described herein. However, those skilled in the art recognize that changes and modifications may be made to the exemplary embodiments without departing from the scope of this disclosure. For example, the various operation steps and the components configured to perform the operation steps can be implemented in different ways according to the specific application or considering any figure of cost functions associated with the operation of the system (for example, one or more steps can be deleted, modified or combined into other steps).

In addition, as understood by those skilled in the art, the principles herein can be reflected in a computer program product on a computer-readable storage medium which is pre-loaded with computer-readable program code. Any tangible, non-transitory computer-readable storage medium can be used, including magnetic storage devices (hard disk, floppy disk, etc.), optical storage devices (CD-ROM, DVD, Blu ray disk, etc.), flash memory and/or the likes. Computer program instructions can be loaded onto a general-purpose computer, a special-purpose computer, or other programmable data processing device to form a machine, so that these instructions executed on a computer or other programmable data processing device can generate a device to realize a specified function. These computer program instructions may also be stored in a computer-readable memory, which may instruct a computer or other programmable data processing device to operate in a specific manner, so that the instructions stored in the computer-readable memory can form a manufactured article, including an implementation device for realizing a specified function. Computer program instructions can also be loaded on a computer or other programmable data processing device, so that a series of operation steps are performed on the computer or other programmable device to generate a computer implemented process, so that the instructions executed on the computer or other programmable device can provide steps for realizing the specified functions.

Although the principles herein have been shown in various embodiments, many modifications of structures, arrangements, proportions, elements, materials, and components that are particularly applicable to specific environmental and operational requirements may be used without departing from the principles and scope of the present disclosure. The above modifications and other changes or amendments are included in the scope of this disclosure.

The foregoing specific description has been described with reference to various embodiments. However, those skilled in the art can recognize that various amendments and changes may be made without departing from the scope of this disclosure. Accordingly, consideration of this disclosure is illustrative rather than restrictive, and all such modifications are included within its scope. Similarly, there are solutions to the advantages, other advantages, and problems of the various embodiments as described above. However, the benefits, advantages, solutions to problems and any solution that can produce these elements or make them more explicit should not be interpreted as critical, necessary or necessary. The term “include” and any other variations thereof as used herein are non-exclusive inclusions, so that a process, method, article or device that includes a list of elements includes not only these elements, but also other elements that are not explicitly listed or do not belong to the process, method, system, article or device. In addition, the term “connection” and any other variation thereof as used herein refer to physical connection, electrical connection, magnetic connection, optical connection, communication connection, functional connection and/or any other connection.

1. An anesthesia control method, comprising:
establishing communication connection between an anesthesia machine and an infusion device;
controlling, through the anesthesia machine, ventilation of a patient according to anesthesia machine control information which is inputted by a user;
sending, through the anesthesia machine, a corresponding infusion device control instruction to the infusion device according to infusion device control information which is inputted by the user; and
through the infusion device, receiving the corresponding infusion device control instruction from the anesthesia machine and controlling medication administration to the patient according to the corresponding infusion device control instruction.

2. The anesthesia control method according to claim 1, wherein the anesthesia machine control information comprises: anesthesia machine control information which is inputted by hand or anesthesia machine control information which is inputted by voice; or
the infusion device control information comprises infusion device control information which is inputted by hand or infusion device control information which is inputted by voice.

3. The anesthesia control method according to claim 1, wherein the communication connection is wireless communication connection or local area network connection.

4. The anesthesia control method according to claim 1, wherein the communication connection is wireless communication connection; and the anesthesia control method further comprises:
through the anesthesia machine, indicating that, the wireless communication connection with the infusion device is established, after establishing the wireless communication connection between the anesthesia machine and the infusion device; or
through the infusion device, indicating that, the wireless communication connection with the anesthesia machine is established, after establishing the wireless communication connection between the anesthesia machine and the infusion device.

5. The anesthesia control method according to claim 1, wherein the anesthesia control method further comprises:
through the infusion device, indicating that the corresponding infusion device control instruction is received, after receiving the corresponding infusion device control instruction, or
through the infusion device, receiving an instruction to close a remote control function and responding to said instruction by stopping receiving the corresponding infusion device control instruction or by stopping responding to the corresponding infusion device control instruction.

6. The anesthesia control method according to claim 1, wherein the communication connection is wireless communication connection and the anesthesia control method further comprises:
through the anesthesia machine, determining whether information of the patient in the anesthesia machine is consistent with information of the patient in the infusion device, before sending the corresponding infusion device control instruction to the infusion device; if not, disconnecting the wireless communication connection, refusing to send the corresponding infusion device control instruction or sending an alarm massage; or
through the infusion device, determining whether information of the patient to whom the infusion device administers medication is consistent with information of the patient whose ventilation is controlled by the anesthesia machine, before controlling the medication administration to the patient according to the corresponding infusion device control instruction; if not, disconnecting the wireless communication connection, refusing to receive or respond to the corresponding infusion device control instruction or sending an alarm massage.

7. (canceled)

8. The anesthesia control method according to claim 1, wherein the infusion device comprises a first injection pump and a second injection pump; and the anesthesia control method further comprises: through the first injection pump, acquiring a working state of the second injection pump when the first injection pump is in a no-output state or a standby state, and before injection, updating information of the patient in the first injection pump at least based on the acquired working state of the second injection pump; or
wherein the infusion device comprises a first injection pump; and the anesthesia control method further comprises: through the first injection pump, acquiring a working state of the anesthesia machine when the first injection pump is in a no-output state or a standby state, and before injection, updating information of the patient in the first injection pump at least based on the acquired working state of the anesthesia machine.

9. (canceled)

10. The anesthesia control method according to claim 8, wherein the anesthesia control method further comprises:
through the anesthesia machine, acquiring a working state of the infusion device when the anesthesia machine is in a no-output state or a standby state, and before ventilation, updating information of the patient in the anesthesia machine at least based on the acquired working state of the infusion device.

11. The anesthesia control method according to claim 1, wherein the anesthesia control method further comprises: displaying, on a same interface of the anesthesia machine, an anesthesia machine setting area for receiving the anesthesia machine control information which is inputted by the user and an infusion device setting area for receiving the infusion device control information which is inputted by the user.

12. An anesthesia control method, comprising:
establishing communication connection between a control terminal and an anesthesia machine, and communication connection between the control terminal and an infusion device;
through the control terminal, sending an anesthesia machine control instruction to the anesthesia machine according to anesthesia machine control information which is inputted by a user, and sending an infusion device control instruction to the infusion device according to infusion device control information which is inputted by the user;
controlling, through the anesthesia machine, ventilation of a patient according to the anesthesia machine control instruction; and
controlling, through the infusion device, medication administration to the patient according to the infusion device control instruction.

13. The anesthesia control method according to claim 12, wherein the anesthesia machine control information comprises: anesthesia machine control information which is inputted by hand or anesthesia machine control information which is inputted by voice; or
the infusion device control information comprises infusion device control information which is inputted by hand or infusion device control information which is inputted by voice.

14. The anesthesia control method according to claim 12, wherein the communication connection is wireless communication connection or local area network connection.

15. The anesthesia control method according to claim 12, wherein the communication connection is wireless communication connection, the control terminal, the anesthesia machine and the infusion device form an anesthesia control system, and the anesthesia control method further comprises:
sending connection indication information after wireless communication connection is established between at least one of the control terminal, the anesthesia machine and the infusion device, and other device(s) in the anesthesia control system.

16. The anesthesia control method according to claim 12, wherein the anesthesia control method further comprises:
through the anesthesia machine, indicating that the anesthesia machine control instruction is received, after receiving the anesthesia machine control instruction; or
through the infusion device, indicating that the infusion device control instruction is received, after receiving the infusion device control instruction.

17. The anesthesia control method according to claim 12, wherein the communication connection is wireless communication connection and the anesthesia control method further comprises:
through the control terminal, determining whether information of the patient in the anesthesia machine is consistent with information of the patient in the control terminal, before sending the anesthesia machine control instruction to the anesthesia machine; if not, disconnecting the wireless communication connection, refusing to send the anesthesia machine control instruction or sending an alarm massage; or
through the control terminal, determining whether information of the patient in the infusion device is consistent with information of the patient in the control terminal, before sending the infusion device control instruction to the infusion device; if not, disconnecting the wireless communication connection, refusing to send the infusion device control instruction or sending an alarm massage; or
through the anesthesia machine, determining whether information of the patient in the control terminal is consistent with information of the patient whose ventilation is controlled by the anesthesia machine, before controlling the ventilation of the patient according to the anesthesia machine control instruction; if not, disconnecting the wireless communication connection, refusing to receive or respond to the anesthesia machine control instruction or sending an alarm massage; or
through the infusion device, determining whether information of the patient to whom the infusion device administers medication is consistent with information of the patient in the control terminal, before the controlling the medication administration to the patient according to the infusion device control instruction; if not, disconnecting the wireless communication connection, refusing to receive or respond to the infusion device control instruction or sending an alarm massage.

18. The anesthesia control method according to claim 12, wherein the anesthesia control method further comprises:
through the anesthesia machine, receiving an instruction to close a remote control function and responding to said instruction by stopping receiving the anesthesia machine control instruction or by stopping responding to the anesthesia machine control instruction; or
through the infusion device, receiving an instruction to close a remote control function and responding to said instruction by stopping receiving the infusion device control instruction or by stopping responding to the infusion device control instruction.

19. The anesthesia control method according to claim 12, wherein the infusion device comprises a first injection pump and a second injection pump; and the anesthesia control method further comprises: through the first injection pump, acquiring a working state of the second injection pump when the first injection pump is in a no-output state or a standby state, and before injection, updating information of the patient in the first injection pump at least based on the acquired working state of the second injection pump; or
wherein the infusion device comprises a first injection pump; and the anesthesia control method further comprises: through the first injection pump, acquiring a working state of the anesthesia machine when the first injection pump is in a no-output state or a standby state, and before injection, updating information of the patient in the first injection pump at least based on the acquired working state of the anesthesia machine.

20. (canceled)

21. The anesthesia control method according to claim 19, wherein the anesthesia control method further comprises:
through the anesthesia machine, acquiring a working state of the infusion device when the anesthesia machine is in a no-output state or a standby state, and before ventilation, updating information of the patient in the anesthesia machine at least based on the acquired working state of the infusion device.

22. The anesthesia control method according to claim 12, wherein the anesthesia control method further comprises: displaying, on a same interface of the control terminal, an anesthesia machine setting area for receiving the anesthesia machine control information which is inputted by the user and an infusion device setting area for receiving the infusion device control information which is inputted by the user.

23. (canceled)

24. An anesthesia control method, comprising:
establishing communication connection between an anesthesia machine and an infusion device;
controlling, through the infusion device, medication administration to a patient according to infusion device control information which is inputted by a user;
sending, through the infusion device, a corresponding anesthesia machine control instruction to the anesthesia machine according to anesthesia machine control information which is inputted by the user; and
through the anesthesia machine, receiving the corresponding anesthesia machine control instruction from the infusion device and controlling ventilation of the patient according to the corresponding anesthesia machine control instruction.

25.-27. (canceled)
